(19) United States
(12) Patent Application Publication
(10) Pub. No.: US 2022/0185746 A1
(43) Pub. Date: Jun. 16, 2022

(54) METHOD FOR PRODUCING 1,3-BUTADIENE

(71) Applicants: JSR Corporation, Tokyo (JP); ENEOS Corporation, Tokyo (JP)

(72) Inventors: Junjie WANG, Tokyo (JP); Takashi MORI, Tokyo (JP); Ryo TANAKA, Tokyo (JP)

(73) Assignees: JSR Corporation, Tokyo (JP); ENEOS Corporation, Tokyo (JP)

(21) Appl. No.: 17/653,167

(22) Filed: Mar. 2, 2022

Related U.S. Application Data
(63) Continuation of application No. PCT/JP2020/032167, filed on Aug. 26, 2020.

(30) Foreign Application Priority Data
Sep. 2, 2019 (JP) 2019-159593

(51) Int. Cl. C07C 5/48

(52) U.S. Cl. CPC C07C 5/48; C07C 2523/31; C07C 2523/28

(57) ABSTRACT

A method for producing 1,3-butadiene, including: (A) performing an oxidative dehydrogenation reaction between oxygen and a raw material gas including n-butene in the presence of a metal oxide catalyst, thereby obtaining a produced gas containing 1,3-butadiene; (B) washing the produced gas obtained in (A); (C) contacting the produced gas washed in (B) with a cooling medium to cool the produced gas; and (D) separating the produced gas cooled in (C) into molecular oxygen and inert gases, and other gases containing 1,3-butadiene, by selective absorption into an absorption solvent. In (B), the washing of the produced gas includes blowing the produced gas onto a liquid surface of a washing liquid so that the produced gas contacts the liquid surface of the washing liquid.

### TECHNICAL FIELD

The present invention relates to a method for producing 1,3-butadiene, and in particular, relates to a method for producing 1,3-butadiene using an oxidative dehydrogenation reaction.

### BACKGROUND ART

Conventionally, as a method for producing 1,3-butadiene (hereinafter also simply referred to as “butadiene”), there has been adopted a method in which components other than butadiene are separated by distillation from a fraction that is obtained by cracking of naphtha and contains molecules with four carbon atoms (hereinafter also referred to as “C4 fraction”).

Demand for butadiene as a raw material for a synthetic rubber or the like has increased, but the amount of C4 fraction supplied has decreased due to a circumstance in which a method for producing ethylene has been changed from a method based on cracking of naphtha to a method based on pyrolysis of ethane. Therefore, production of butadiene in which the C4 fraction is not adopted as a raw material is required.

Regarding methods for producing butadiene, attention has been paid to a method in which butadiene is isolated and obtained from a produced gas that has been obtained by oxidative dehydrogenation of n-butene (for example, see Patent Literatures 1 to 4). This production method includes an oxidative dehydrogenation step of performing an oxidative dehydrogenation reaction of a raw material gas containing n-butene and a molecular oxygen-containing gas containing molecular oxygen (for example, air), a cooling step of cooling a produced gas obtained in the previous step, and a produced gas separating step of isolating butadiene from the produced gas cooled in the previous step. In the cooling step, a quench tower in which the produced gas is quenched by countercurrent contact with a cooling medium and which includes, for example, a packing tower or a tray tower is used.

### CITATION LIST

### Patent Literature

Patent Literature 1: Japanese Translation of PCT Patent Application Publication No. 2016-503072

Patent Literature 2: Japanese Translation of PCT Patent Application Publication No. 2019-501938

Patent Literature 3: Japanese Patent Application Laid-Open No. 2013-119530

Patent Literature 4: International Publication No. 2018/234158

### SUMMARY OF INVENTION

### Technical Problem

However, in the aforementioned method for producing butadiene, reaction by-products such as carbonyl compounds, which include acetaldehyde, methyl vinyl ketone and others, and organic acids, which include carboxylic acid and others, are generated. Such reaction by-products are deposited, for example, by quenching the produced gas in the cooling step, and a solid component is attached to a solid packing or a tray in the quench tower used. When the amount of a solid component attached increases with time, a differential pressure is generated in the quench tower making continuance of the cooling step impossible. Therefore, disassembly of the quench tower and removal of the attached solid component are required, and it is difficult to stably cool the produced gas for an extended period of time.

The present invention has been made in view of the foregoing circumstances and has as its object the provision of a method for producing 1,3-butadiene in which generation of a solid component in a step of cooling a produced gas can be prevented or suppressed, and the produced gas can be cooled stably for an extended period of time.

### Solution to Problem

A method for producing 1,3-butadiene of the present invention includes:

a step (A) of performing an oxidative dehydrogenation reaction, with oxygen in the presence of a metal oxide catalyst, of a raw material gas, which contains n-butene, to obtain a produced gas containing 1,3-butadiene;

a step (B) of washing the produced gas obtained in the step (A);

a step (C) of bringing the produced gas, which has been washed in the step (B), into contact with a cooling medium to cool the produced gas; and

a step (D) of separating the produced gas, which has been cooled in the step (C), into molecular oxygen and inert gases, and other gases containing 1,3-butadiene, by selective absorption into an absorption solvent, wherein

in the step (B), washing the produced gas includes blowing the produced gas onto and bringing it into contact with a liquid surface of a washing liquid.

In the method for producing 1,3-butadiene of the present invention, it is preferable that the step (B) is performed in a washing unit and that the mass ratio of the washing liquid to the produced gas in the washing unit is 1.1 to 8.9.

In the method for producing 1,3-butadiene of the present invention, it is preferable that the washing unit has one or more stages that include scrubbing parts for washing the produced gas.

In the method for producing 1,3-butadiene of the present invention, it is preferable that the pH of the washing liquid in the step (B) is within a range of 6 to 10.

In the method for producing 1,3-butadiene of the present invention, it is preferable that the washing liquid after washing the produced gas in the step (B) is used as a cooling medium for cooling the produced gas in the step (C).

### Advantageous Effects of Invention

According to the method for producing 1,3-butadiene of the present invention, before the step (C) of cooling the produced gas, the produced gas is washed in the step (B) to remove a solid component which would be deposited. Therefore, attachment of a large amount of the solid component, which would be deposited by cooling in the step (C), to a solid packing, a tray and the like can be prevented or suppressed. Accordingly, the produced gas can be cooled stably for an extended period of time.

### BRIEF DESCRIPTION OF DRAWINGS

FIG. 1 is a flow diagram illustrating an example of a specific procedure for performing a method for producing butadiene of the present invention.

FIG. 2 is an explanatory diagram illustrating a configuration of an example of a washing unit which can be used in a step (B) in the present invention.

FIG. 3 is an explanatory diagram illustrating a configuration of an example of a quench tower which can be used in a step (C) in the present invention.

### DESCRIPTION OF EMBODIMENTS

Hereinafter, embodiments of the present invention will be described.

A method for producing butadiene (1,3-butadiene) of the present invention has steps shown in (1) to (4) below, and is to produce butadiene from a raw material gas, which contains n-butene, by performing the steps (1) to (4) described below.

(1) A step (A) of performing an oxidative dehydrogenation reaction, with oxygen in the presence of a metal oxide catalyst, of a raw material gas, which contains n-butene, to obtain a produced gas containing 1,3-butadiene;

(2) A step (B) of washing the produced gas obtained in the step (A);

(3) A step (C) of bringing the produced gas, which has been washed in the step (B), into contact with a cooling medium, to cool the produced gas; and

(4) A step (D) of separating the produced gas, which has been cooled in the step (C), into molecular oxygen and inert gases, and other gases containing 1,3-butadiene by selective absorption into an absorption solvent.

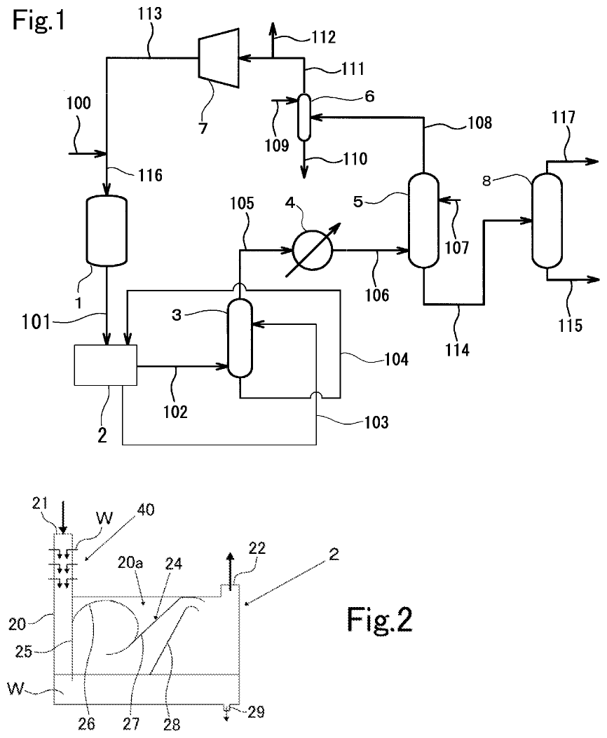

FIG. 1 is a flow diagram illustrating an example of a specific procedure for performing the method for producing butadiene of the present invention. Hereinafter, a specific example of the method for producing butadiene of the present invention will be described in detail using FIG. 1.

The method for producing butadiene exemplified in FIG. 1 includes, in addition to the steps of (1) to (4), a desolvation step of separating an absorption solvent, which has absorbed the other gases containing 1,3-butadiene obtained in the step (D), and a circulation step of returning the molecular oxygen and the inert gases obtained in the step (D) to the step (A), that is, feeding them as a reflux gas.

The absorption solvent used in the step (D) is circulated and used in the method for producing butadiene exemplified in FIG. 1.

Step (A):

In the step (A), the raw material gas and the molecular oxygen-containing gas are subjected to an oxidative dehydrogenation reaction in the presence of a metal oxide catalyst to obtain a produced gas containing butadiene (1,3-butadiene). In this step (A), the oxidative dehydrogenation reaction, with the molecular oxygen-containing gas, of the raw material gas is performed in a reactor 1 as illustrated in FIG. 1. Herein, the reactor 1 is a tower-shaped reactor which has a gas inlet provided at an upper portion and a gas outlet provided at a lower portion and includes a catalyst layer (not illustrated in the drawing) formed by filling the inside of the reactor with the metal oxide catalyst. Pipings 100 and 113 are connected to the gas inlet of the reactor 1 via a piping 116, and a piping 101 is connected to a gas outlet of a washing unit 2.

The step (A) will specifically be described. The raw material gas and the molecular oxygen-containing gas, and, as necessary, inert gases and water (water vapor) (hereinafter also collectively referred to as “newly supplied gas”) are supplied to the reactor 1 through the piping 100 that communicates with the piping 116. The newly supplied gas is heated, before introduction into the reactor 1, to a temperature between about 200° C. and about 400° C. by a preheater (not illustrated) which is disposed between the reactor 1 and the piping 100. Together with the newly supplied gas, which is supplied through the piping 100, the reflux gas from the circulation step, after being heated by the preheater, is supplied to the reactor 1 through the piping 113 that communicates with the piping 116. That is, a mixed gas including the newly supplied gas and the reflux gas is supplied to the reactor 1 after being heated by the preheater. Herein, the newly supplied gas and the reflux gas may each be supplied directly to the reactor 1 through separate pipings. However, it is preferable that the newly supplied gas and the reflux gas in a mixed state are supplied to the reactor 1 through the common piping 116, as illustrated in FIG. 1. When the common piping 116 is provided, a mixed gas that contains various components and is in a state where the components are uniformly mixed in advance can be supplied to the reactor 1. This can prevent a situation where a gas mixed in a nonuniform manner partially forms a detonating gas in the reactor 1.

In the reactor 1 to which the mixed gas has been supplied, butadiene (1,3-butadiene) is produced by an oxidative dehydrogenation reaction, with the molecular oxygen-containing gas, of the raw material gas. Thus, a produced gas containing the butadiene is obtained. The obtained produced gas is discharged from the gas outlet of the reactor 1 to the piping 101.

Raw Material Gas:

As the raw material gas, a gaseous substance obtained by gasifying n-butene, which is a monoolefin having 4 carbon atoms, by a vaporizer (not illustrated) is used. This raw material gas is a combustible gas. In the present invention, “n-butene” means linear butenes, and specifically, may include 1-butene, cis-2-butene and trans-2-butene.

In addition, optional impurities may be contained in the raw material gas. Specific examples of the impurities include branched monoolefins such as i-butene, and saturated hydrocarbons such as propane, n-butane and i-butane. In addition, the raw material gas may contain 1,3-butadiene as an impurity, which is the production target. The amount of impurities contained in the raw material gas is usually not more than 60% by volume, and may preferably be not more than 40% by volume, more preferably not more than 25% by volume, particularly preferably not more than 5% by volume, per 100% by volume of the raw material gas. When the amount of impurities is excessively large, the reaction rate tends to be lowered due to the decrease in the concentration of linear butene in the raw material gas, or the amount of by-products tends to increase.

As the raw material gas, for example, a fraction (raffinate 2) containing a linear butene, as a main component, obtained by separating butadiene and i-butene from a C4 fraction (a fraction containing molecules with 4 carbon atoms), which is by-produced by naphtha cracking, or a butene fraction generated by a dehydrogenation reaction or an oxidative dehydrogenation reaction of n-butane may be used. High purity 1-butene, cis-2-butene and trans-2-butene, which are obtained by dimerization of ethylene, and gas mixtures thereof may also be used. In addition, gasses containing a large amount of hydrocarbons with 4 carbon atoms (hereinafter, sometimes abbreviated as “FCC-C4”) may be used as raw material gases as they are, and here the gasses can be obtained through Fluid Catalytic Cracking by cracking a heavy oil fraction, which is obtained when a crude oil is distilled in a petroleum refining plant or the like, using a powdery solid catalyst in a fluidized bed state to convert the heavy oil fraction into hydrocarbons having low boiling points. Gasses obtained by removing impurities such as phosphorus from FCC-C4 may also be used as a raw material gas.

Molecular Oxygen-Containing Gas:

The molecular oxygen-containing gas is usually a gas containing 10 volume % or more of molecular oxygen (O₂). In this molecular oxygen-containing gas, the concentration of molecular oxygen may preferably be not less than 15% by volume, more preferably not less than 20% by volume.

The molecular oxygen-containing gas may include, in addition to molecular oxygen, an optional gas such as molecular nitrogen (N₂), argon (Ar), neon (Ne), helium (He), carbon monoxide (CO), carbon dioxide (CO₂) and water (water vapor). The amount of the optional gas in the molecular oxygen-containing gas is usually not more than 90% by volume, and may be preferably not more than 85% by volume, more preferably not more than 80% by volume when the optional gas is molecular nitrogen, and is usually not more than 10% by volume, and may preferably be not more than 1% by volume when the optional gas is a gas other than molecular nitrogen. When the amount of the optional gas is excessively large, in the reaction system (inside of the reactor 1), molecular oxygen of a required amount may not coexist with the raw material gas. In the step (A), preferred specific examples of the molecular oxygen-containing gas include air.

Inert Gases:

It is preferable that inert gases are supplied to the reactor 1 together with the raw material gas and the molecular oxygen-containing gas.

When inert gases are supplied to the reactor 1, the concentrations (relative concentrations) of the raw material gas and the molecular oxygen can be adjusted in such a manner that the mixed gas does not form a detonating gas in the reactor 1.

Examples of the inert gases include molecular nitrogen (N₂), argon (Ar) and carbon dioxide (CO₂). These may be used either singly or in any combination of two or more thereof. Among these, molecular nitrogen is preferred from an economic viewpoint.

Water (Water Vapor):

It is preferable that water is supplied to the reactor 1 together with the raw material gas and the molecular oxygen-containing gas.

By supplying water to the reactor 1, it is possible to adjust the concentrations (relative concentrations) of the raw material gas and the molecular oxygen in the same manner as that in the aforementioned inert gases in such a manner that the mixed gas does not form a detonating gas in the reactor 1.

Mixed Gas:

Since the mixed gas contains the combustible raw material gas and the molecular oxygen, the composition thereof is adjusted in such a manner that the concentration of the raw material gas does not fall within an explosive range.

Specifically, the composition of the mixed gas at the gas inlet of the reactor 1 is controlled by monitoring the flow rates with flow meters (not illustrated) installed in the pipings (specifically the piping (not illustrated) communicating with the piping 100, and the piping 113) for supplying respective gases constituting the mixed gas (specifically, the raw material gas, molecular oxygen-containing gas (air), and inert gasses and water (water vapor) used as necessary) to the reactor 1. For example, depending on the molecular oxygen concentration of the reflux gas supplied to the reactor 1 through the piping 113, the composition of the newly supplied gas to be supplied to the reactor 1 through the piping 100 is controlled.

In this specification, the “explosive range” indicates a range in which the mixed gas has a composition such that it ignites in the presence of some ignition source. Here, it is known that an ignition source that coexists does not ignite a combustible gas when the concentration of the combustible gas is lower than a certain value, and this concentration is referred to as a lower explosion limit. Such a lower explosion limit is the lower limit of the explosive range. It is also known that, when the concentration of the combustible gas is higher than a certain value, an ignition source that coexists does not ignite the combustible gas, and this concentration is referred to as an upper explosion limit. Such an upper explosion limit is the upper limit of the explosive range. These values depend on the concentration of molecular oxygen. In general, the lower the concentration of molecular oxygen is, the more the values approach each other. When the concentration of molecular oxygen becomes a certain value, the values coincide with each other. The concentration of molecular oxygen at this time is referred to as the limit oxygen concentration. Thus, if the concentration of molecular oxygen in the mixed gas is lower than the limit oxygen concentration, the mixed gas does not ignite regardless of the concentration of the raw material gas.

Specifically, in the mixed gas, the concentration of the sum of 1-butene and 2-butene may preferably be not less than 2% by volume and not more than 30% by volume, more preferably not less than 3% by volume and not more than 25% by volume, particularly preferably not less than 5% by volume and not more than 20% by volume, per 100% by volume of the mixed gas from the viewpoint of productivity of butadiene and suppression of burden on the metal oxide catalyst. If the concentration of the sum of 1-butene and 2-butene is excessively low, the productivity of butadiene may decrease. On the other hand, when the concentration of the sum of 1-butene and 2-butene is excessively large, the burden on the metal oxide catalyst may increase.

The concentration (relative concentration) of the molecular oxygen relative to the raw material gas in the mixed gas is preferably not less than 50 parts by volume and not more than 170 parts by volume, more preferably not less than 70 parts by volume and not more than 160 parts by volume, per 100 parts by volume of the raw material gas. When the concentration of the molecular oxygen in the mixed gas is out of the aforementioned range, there is a tendency in which the concentration of the molecular oxygen at the gas outlet of the reactor 1 is difficult to be adjusted by adjusting the reaction temperature. Since the concentration of the molecular oxygen at the gas outlet of the reactor 1 cannot be controlled by the reaction temperature, decomposition of target product and occurrence of side reaction inside the reactor 1 may not be suppressed.

The concentration (relative concentration) of the molecular nitrogen relative to the raw material gas in the mixed gas is preferably not less than 400 parts by volume and not more than 1,800 parts by volume, more preferably not less than 500 parts by volume and not more than 1,700 parts by volume, per 100 parts by volume of the raw material gas. The concentration (relative concentration) of water (water vapor) relative to the raw material gas is preferably 0 part by volume or more and not more than 900 parts by volume, more preferably not less than 80 parts by volume and not more than 300 parts by volume, per 100 parts by volume of the raw material gas. When either the concentration of the molecular nitrogen or the concentration of water is excessively high, the concentration of the raw material gas is decreased with an increase in the concentration of the molecular nitrogen or the concentration of water. Therefore, there is a tendency in which the production efficiency of butadiene decreases. In contrast, when either the concentration of the molecular nitrogen or the concentration of water is excessively low, there is a tendency in which the concentration of the raw material gas falls within an explosive range with a decrease in the concentration of the molecular nitrogen or the concentration of water or removal of heat in a reaction system as described below is difficult.

Metal Oxide Catalyst:

As the metal oxide catalyst, a composite oxide catalyst containing molybdenum and bismuth is used. As such a composite oxide catalyst, for example, those containing at least molybdenum (Mo), bismuth (Bi) and iron (Fe) can be used, and specific examples thereof include those containing a composite metal oxide represented by the following composition formula (1).

MoₐBi_{b}Fe_{c}X_{d}YₑZ_{f}O_{g} Composition formula (1):

In the aforementioned composition formula (1), X is at least one selected from the group consisting of Ni and Co. Y is at least one selected from the group consisting of Li, Na, K, Rb, Cs and Tl. Z is at least one selected from the group consisting of Mg, Ca, Ce, Zn, Cr, Sb, As, B, P and W. a, b, c, d, e, f and g each independently show the atomic ratio of each element; when a is 12, b is 0.1 to 8, c is 0.1 to 20, d is 0 to 20, e is 0 to 4, f is 0 to 2, and g is the number of atoms of the oxygen element required to satisfy the atomic valence of each of the aforementioned components.

A composite oxide catalyst containing the composite metal oxide represented by the aforementioned composition formula (1) is highly active and highly selective in a production method of butadiene using an oxidative dehydrogenation reaction, and is further excellent in life stability.

The method for preparing the composite oxide catalyst is not particularly limited, and a known method such as an evaporation and drying method, a spray drying method, or an oxide mixing method using raw materials of respective elements relating to the composite metal oxide constituting the composite oxide catalyst to be prepared can be adopted.

The raw materials of the aforementioned respective elements are not particularly limited, and examples thereof include an oxide, a nitrate salt, a carbonate salt, an ammonium salt, a hydroxide, a carboxylic acid salt, an ammonium carboxylate salt, an ammonium halide salt, a hydrogenated acid and an alkoxide of the component elements.

Furthermore, the composite oxide catalyst may be used while being carried on an inert carrier. Examples of the carrier species include silica, alumina and silicon carbide.

Oxygen Dehydrogenation Reaction:

When an oxidative dehydrogenation reaction is initiated in the step (A), it is preferable that supply of the molecular oxygen-containing gas, inert gases and water (water vapor) to the reactor 1 is first initiated, then the amounts supplied of these are adjusted so that the concentration of molecular oxygen at the gas inlet of the reactor 1 is not more than the limit oxygen concentration, and supply of the raw material gas is initiated next, and then the amount supplied of the raw material gas and the amount supplied of the molecular oxygen-containing gas are increased so that the concentration of the raw material gas at the gas inlet of the reactor 1 exceeds the upper explosion limit.

When the amounts supplied of the raw material gas and the molecular oxygen-containing gas are increased, the amount supplied of the mixed gas may be made constant by decreasing the amount supplied of water (water vapor). Thus, the time the gas resides in the pipings and the reactor 1 can be kept constant, and a change in pressure in the reactor 1 can be suppressed.

The pressure in the reactor 1 (specifically, the pressure at the gas inlet of the reactor 1) that is the pressure in the step (A) may preferably be not less than 0.1 MPaG and not more than 0.4 MPaG, more preferably not less than 0.15 MPaG and not more than 0.35 MPaG, further preferably not less than 0.2 MPaG and not more than 0.3 MPaG.

When the pressure in the step (A) is controlled to fall within the aforementioned range, the reaction efficiency in the oxidative dehydrogenation reaction improves.

When the pressure in the step (A) is excessively low, the reaction efficiency in the oxidative dehydrogenation reaction tends to decrease. On the other hand, when the pressure in the step (A) is excessively high, the yield in the oxidative dehydrogenation reaction tends to decrease.

In the oxidative dehydrogenation reaction, a gas hourly space velocity (GHSV) determined by the following expression (1) may preferably be not less than 500 h⁻¹ and not more than 5,000 h⁻¹, more preferably not less than 800 h⁻¹ and not more than 3,500 h⁻¹, further preferably not less than 1,000 h⁻¹ and not more than 3,000 h⁻¹.

When GHSV is controlled to fall within the aforementioned range, the reaction efficiency in the oxidative dehydrogenation reaction can improve further.

GHSV [h⁻¹]=gas flow rate at atmospheric pressure [Nm³/h]/catalyst layer volume [m³] Expression (1):

The “catalyst layer volume” in the aforementioned expression (1) represents a volume (apparent volume) of the whole catalyst layer containing pores.

In the oxidative dehydrogenation reaction, the gas hourly space velocity based on an actual volume (GHSV based on actual volume) determined by the following expression (2) is preferably not less than 500 h⁻¹ and not more than 2,300 h⁻¹, more preferably not less than 600 h⁻¹ and not more than 2,000 h⁻¹, further preferably not less than 700 h⁻¹ and not more than 1,500 h⁻¹.

When GHSV based on actual volume is controlled to fall within the aforementioned range, the reaction efficiency in the oxidative dehydrogenation reaction can improve further.

GHSV based on actual volume [h⁻¹]=actual gas flow rate [m³/h]/catalyst layer volume [m³] Expression (2):

The “catalyst layer volume” in the aforementioned expression (2) represents a volume (apparent volume) of the whole catalyst layer containing pores, similarly to the expression (1).

Since an oxidative dehydrogenation reaction is an exothermic reaction, the temperature of a reaction system in the oxidative dehydrogenation reaction increases, and a plurality of kinds of by-products may be produced. As the by-products, unsaturated carbonyl compounds with 3 to 4 carbon atoms such as acrolein, acrylic acid, methacrolein, methacrylic acid, maleic acid, fumaric acid, maleic anhydride, methyl vinyl ketone, crotonaldehyde and crotonic acid are produced, and the concentration of the by-products in the produced gas increases, causing various adverse influences. Specifically, the aforementioned unsaturated carbonyl compounds are dissolved in the absorption solvent and the like that are circulated and used in the step (D). Thus, impurities easily accumulate in the absorption solvent and the like, and deposition of a deposit on each member is easily induced.

As examples of a procedure for controlling the concentration of the unsaturated carbonyl compound to fall within a certain range in the oxidative dehydrogenation reaction, may be mentioned a method for adjusting the reaction temperature of the oxidative dehydrogenation reaction. When the reaction temperature is adjusted, the concentration of molecular oxygen at the gas outlet of the reactor 1 can be within a certain range.

Specifically, the reaction temperature may preferably be not lower than 300° C. and not higher than 400° C., more preferably not lower than 320° C. and lower than 380° C.

When the reaction temperature is controlled to fall within the aforementioned range, coking (deposition of solid carbon) can be suppressed in the metal oxide catalyst, and the concentration of the unsaturated carbonyl compound in the produced gas can be within a certain range. Furthermore, the concentration of molecular oxygen at the gas outlet of the reactor 1 can also be within a certain range.

On the other hand, when the reaction temperature is excessively low, the conversion rate of n-butene may decrease. When the reaction temperature is excessively high, the concentration of the unsaturated carbonyl compound increases, and there is a tendency for an impurity to accumulate in the absorption solvent and the like, or coking to occur in the metal oxide catalyst.

Herein, as specific preferable examples of the method for adjusting the reaction temperature, may be mentioned a procedure in which the reactor 1 is appropriately cooled by removing heat with a heating medium (specifically, dibenzyltoluene, a nitrite salt or the like), to control the temperature of the catalyst layer to be constant.

Produced Gas:

The produced gas includes reaction by-products, the raw material gas unreacted, molecular oxygen unreacted, the gas for concentration adjustment and others in addition to 1,3-butadiene, which is a target product of the oxidative dehydrogenation reaction of the raw material gas and the molecular oxygen-containing gas. Examples of the reaction by-products include a carbonyl compound and a heterocyclic compound. Herein, the carbonyl compound includes ketones, aldehydes and organic acids.

As examples of the ketones, may be mentioned methyl vinyl ketone, acetophenone, benzophenone, anthraquinone and fluorenone.

As examples of the aldehydes, may be mentioned acetaldehyde, acrolein, methacrolein, crotonaldehyde and benzaldehyde.

As examples of the organic acids, may be mentioned maleic acid, fumaric acid, acrylic acid, phthalic acid, benzoic acid, crotonic acid, tetrahydrophthalic acid, isophthalic acid, terephthalic acid, methacrylic acid and phenol.

As examples of the heterocyclic compound, furan may be mentioned.

Step (B):

In the step (B), the produced gas obtained in the step (A) is washed by blowing the produced gas onto and bringing it into contact with a liquid surface of a washing liquid. It is preferable that the step (B) is performed in the washing unit 2 which has one or more stages that include scrubbing parts for washing the produced gas.

FIG. 2 is an explanatory diagram illustrating a configuration of an example of a washing unit that can be used in the step (B). In this washing unit 2, a scrubbing part 20a is provided in the inside of a chamber 20 which has a gas inlet 21 for introducing the produced gas from the step (A) and a gas outlet 22 for discharging the produced gas that has been washed. Inside the chamber 20, a washing liquid W is stored at the bottom, and a washing-liquid outlet 29 for discharging the washing liquid W outside is provided at the bottom wall. This washing-liquid outlet 29 is connected to a piping 103.

The scrubbing part 20a has a flow channel-forming member 24 for forming a flow channel for the produced gas.

In the scrubbing part 20a, the flow channel-forming member 24 includes a vertical plate 25 that is provided opposite to a side wall of the chamber 20, a first curved plate 26 that is provided on a back surface of the vertical plate 25 and is curved in a partial elliptical shape, a second curved plate 27 that is provided on a back surface of the first curved plate 26, and an auxiliary guide plate 28 that is provided opposite to the second curved plate 27. The vertical plate 26 is provided so that the lower end thereof is dipped in the stored washing liquid W. Between the side wall of the chamber 20 and the vertical plate 25, a flow channel that guides the produced gas introduced from the gas inlet 21 to the stored washing liquid W is formed. Between the second curved plate 27 and the auxiliary guide plate 28, a flow channel with a width that gradually decreases from an upstream side toward a downstream side is formed.

In the aforementioned washing unit 2, the produced gas from the step (a) is introduced from the gas inlet 21 to the scrubbing part 20a. The introduced produced gas passes through the flow channel between the vertical plate 25 and the side wall of the chamber 20 downwards and together with the washing liquid W which is sprayed from a washing-liquid sprayer 40. The produced gas is blown onto and brought into contact with the liquid surface of the stored washing liquid W, and as a result, a part of the by-products in the produced gas is deposited in the washing liquid W as a solid component. When the produced gas is blown onto the liquid surface of the stored washing liquid W, pressure is applied to push down the liquid surface of the washing liquid W, and a slight space is formed between the lower end of the vertical plate 25 and the liquid surface of the washing liquid W. When the produced gas passes through this space, droplets of the washing liquid W spurt out due to the Venturi effect. At this time, the pressure in the first curved plate 26 becomes negative, generating a vortex flow. Thus, the deposited solid component is sucked in and collected together with fine droplets of the washing liquid W. When the produced gas passes, together with droplets with a relatively large particle size of the washing liquid W, through the flow channel between the second curved plate 27 and the auxiliary guide plate 28, droplets of the washing liquid W is separated from the produced gas due to abrupt deceleration of passing speed and slow directional change of the flow of the produced gas. Thus, the produced gas is washed at the scrubbing part 20a, and the produced gas is then discharged from the gas outlet 22 to the outside. A part of the stored washing liquid W is discharged from the washing-liquid outlet 29 outside.

In the step (B) with such a configuration, water or alkaline water can be used as the washing liquid W. The temperature of the washing liquid W may preferably be not lower than 5° C. and not higher than 50° C., more preferably not lower than 5° C. and not higher than 30° C.

The washing liquid W that has been discharged from the washing-liquid outlet 29 may be discarded, but it is preferable that the washing liquid W is used as all or a part of a cooling medium in the step (C) described below. In the illustrated example, the washing liquid W that has been discharged from the washing-liquid outlet 29 is supplied, through the piping 103, to a mechanism for supplying a cooling medium 38 in a quench tower 3 and is reused as the cooling medium in the step (C).

It is preferable that the mass ratio of the washing liquid W to the produced gas (washing liquid/produced gas) in the washing unit 2 is 1.1 to 8.9. When this mass ratio is less than 1.1, washing of the gas is insufficient, and dirt may accumulate in a member for gas-liquid contact 37 inside the quench tower 3 in the step (C) described below. In contrast, when this mass ratio is more than 8.9, a balance between the washing liquid and the produced gas is lost and a washing mechanism due to the Venturi effect cannot be maintained. Thus, the controllability of the washing unit 2 may deteriorate, and similarly, dirt may accumulate also in the member for gas-liquid contact 37 inside the quench tower 3 in the step (C) described below.

In the step (B), it is preferable that the pH of the washing liquid W is within a range of 6 to 10. When the pH of the washing liquid W is less than 6, a dirt-cleaning effect of the washing liquid is not exerted, and dirt may accumulate in the member for gas-liquid contact 37 inside the quench tower 3 in the step C described below. In contrast, when the pH of the washing liquid W is more than 10, there is a concern about alkali corrosion having an influence on equipment.

Step (C):

In the step (C), the produced gas that has been washed in the step (B) is cooled. In the step (C) exemplified in the drawings, cooling of the produced gas from the step (B) is performed by the quench tower 3 and a heat exchanger for cooling 4.

The step (C) will specifically be described. The produced gas from the step (B) is fed to the quench tower 3 through a piping 102, and is cooled by the quench tower 3. Then, the produced gas is fed to the heat exchanger for cooling 4 through a piping 105, and is further cooled by the heat exchanger for cooling 4. The produced gas from the step (A), which has thus been cooled by the quench tower 3 and the heat exchanger for cooling 4, is discharged from the heat exchanger for cooling 4 to a piping 106.

The produced gas from the step (A) is purified by undergoing this step (C). Specifically, a part of the by-products contained in the produced gas from the step (A) is condensed or deposited by cooling and is then removed.

Quench Tower:

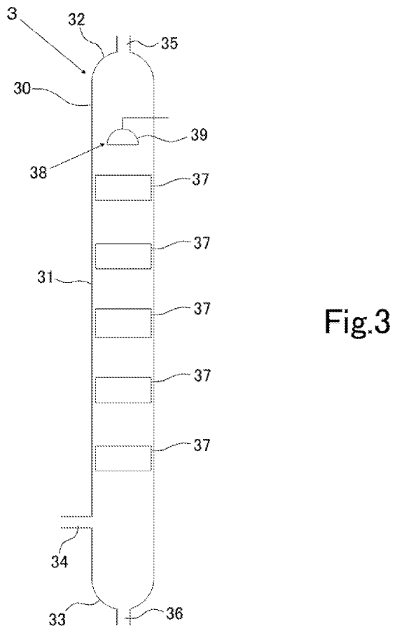

FIG. 3 is an explanatory diagram illustrating a configuration of an example of a quench tower that can be used in the step (C). The quench tower 3 is configured to bring the produced gas into countercurrent contact with the cooling medium in order to quench the produced gas, for example, to a temperature between about 30° C. and about 90° C. Also, the quench tower 3 has a cylindrical tower body 30 that extends in the vertical direction and has both closed ends.

A gas inlet 34 for introducing the produced gas from the step (B) into the tower body 30 is provided at a lower portion of a peripheral wall portion 31 of the tower body 30. A gas outlet 35 for discharging the cooled produced gas from the inside of the tower body 30 is provided at a tower top 32 of the tower body 30. A cooling-medium outlet 36 for discharging the cooling medium supplied to the inside of the tower body is provided at a tower bottom 33 of the tower body 30.

The member for gas-liquid contact 37 is provided between the gas inlet 34 and the gas outlet 35 in the tower body 30. In the illustrated example, a plurality of the members for gas-liquid contact 37 are arranged so that the members overlap each other in the vertical direction, with a space interposed therebetween. The mechanism for supplying a cooling medium 38 which supplies a cooling medium for cooling a produced gas is provided at a position above an uppermost member for gas-liquid contact 37.

When a packing tower is adopted as the quench tower 3, a random packing can be used as the member for gas-liquid contact 37. As specific examples of the random packing, may be mentioned raschig rings, pall-rings and cascade mini rings. When a tray tower is adopted as the quench tower 3, a tray is used as the member for gas-liquid contact 37.

The mechanism for supplying a cooling medium 38 has a spraying head 39 for spraying a cooling medium. This spraying head 39 is disposed so as to face downwards and is positioned above the uppermost member for gas-liquid contact 37 in the tower body 30. As the cooling medium supplied from the mechanism for supplying a cooling medium 38, water or alkaline water can be used. The temperature of the cooling medium is appropriately determined depending on the cooling temperature of the produced gas. The temperature of the cooling medium may preferably be not lower than 10° C. and not higher than 90° C., more preferably not lower than 20° C. and not higher than 70° C., particularly preferably not lower than 20° C. and not higher than 40° C.

The piping 102, which has an end connected to the gas outlet 22 of the reactor 1, is connected to the gas inlet 34 of the quench tower 3, and the piping 105 is connected to the gas outlet 35 of the quench tower 3. The piping 103 is connected to the spraying head 39 of the mechanism for supplying a cooling medium 38 in the quench tower 3, and a piping 104 is connected to the cooling-medium outlet 36 of the quench tower 3.

The temperature of the inside of the quench tower 3 during operation may preferably be not lower than 10° C. and not higher than 100° C., more preferably not lower than 20° C. and not higher than 90° C.

It is preferable that the pressure of the quench tower 3 during operation (specifically, the pressure at the gas outlet of the quench tower 3), that is, the pressure in the step (C) is equal to or less than the pressure in the step (A).

Specifically, the difference in pressure between the steps (C) and (A), that is, a value obtained by subtracting the pressure in the step (C) from the pressure in the step (A) may preferably be 0 MPaG or more and not more than 0.05 MPaG, more preferably not less than 0.01 MPaG and not more than 0.04 MPaG.

When the difference in pressure between the steps (A) and (C) is controlled to fall within the aforementioned range, both the condensation of reaction by-products in the produced gas from the step (A) and the dissolution of the reaction by-products in the cooling medium can be promoted, and as a result, the concentration of the reaction by-products can be decreased in the produced gas that is discharged from the quench tower 3.

In the aforementioned quench tower 3, the produced gas is introduced from the gas outlet 34 into the tower body 30 and discharged from the gas outlet 35. Thus, the produced gas is passed from the lower portion of the tower body 30 through the members for gas-liquid contact 37 to the upper portion of the tower body 30 in the tower body 30. On the other hand, the cooling medium from the mechanism for supplying a cooling medium 38 is sprayed downward from the spraying head 39. Thus, the produced gas that is flowing upward in the inside of the tower body 30 is brought into countercurrent contact with the cooling medium, and as a result, the produced gas is quenched. The supplied cooling medium is discharged from the cooling-medium outlet 36 to the outside of the tower body 30. The discharged cooling medium maybe discarded or used as the washing liquid W in the step (B). In the illustrated example, the cooling medium that is discharged from the cooling-medium outlet 36 is supplied to the washing-liquid sprayer 40 in the washing unit 2 through the piping 104 and used again as the washing liquid W in the step (B).

Heat Exchanger for Cooling:

As the heat exchanger for cooling 4, a heat exchanger that is capable of cooling the produced gas, which has been discharged from the quench tower 3, to room temperature (not lower than 10° C. and not higher than 30° C.) is appropriately used.

In the illustrated example, the heat exchanger for cooling 4 has a gas inlet, which the piping 105 having an end connected to the gas outlet 35 of the quench tower 3 is connected to, and a gas outlet, which the piping 106 is connected to.

It is preferable that the pressure of the heat exchanger for cooling 4 during operation (specifically, the pressure at the gas outlet of the heat exchanger for cooling 4) is equal to the pressure of the quench tower 3 during operation (the pressure at the gas outlet of the quench tower 3).

The concentration of molecular nitrogen in the cooled produced gas discharged from the heat exchanger for cooling 4, that is, in the produced gas cooled in the step (C) may preferably be not less than 60% by volume and not more than 94% by volume, more preferably not less than 70% by volume and not more than 85% by volume. The concentration of butadiene may preferably be not less than 2% by volume and not more than 15% by volume, more preferably not less than 3% by volume and not more than 10% by volume. The concentration of water (water vapor) may preferably be not less than 1% by volume and not more than 30% by volume, more preferably not less than 1% by volume and not more than 3% by volume. The concentration of the ketones and aldehydes may preferably be 0% by volume or more and not more than 0.3% by volume, more preferably not less than 0.05% by volume and not more than 0.25% by volume.

When the concentration of each component in the produced gas cooled in the step (C) is controlled to fall within the abovementioned range, the efficiency of butadiene purification in the next and subsequent steps can be improved and side reactions occurring in the desolvation step can be suppressed. This allows the energy consumption in the production of butadiene to be further reduced.

Step (D):

In the step (D), the produced gas that has been subjected to the step (C) is separated (roughly separated) into molecular oxygen and inert gases, and the other gases containing 1,3-butadiene by selective absorption into the absorption solvent. Herein, the “other gases containing 1,3-butadiene” refer to at least a gas containing at least butadiene, and 1-butene and 2-butene (unreacted 1-butene and unreacted 2-butene), which are absorbed by the absorption solvent.

In this step (D), the separation of the cooled produced gas, which has been subjected to the step (C), is performed by an absorption tower 5, as illustrated in FIG. 1. Herein, the absorption tower 5 is a tower in which a gas inlet for introducing the produced gas, which has been subjected to the step (C), is provided at a lower portion, a solvent inlet for introducing the absorption solvent is provided at an upper portion, a solvent outlet for discharging the absorption solvent, which gases (specifically, the other gases containing 1,3-butadiene) are absorbed in, is provided at a tower bottom, and a gas outlet 35 for discharging a gas that has not been absorbed by the absorption solvent (specifically, molecular oxygen and inert gases) is provided at a tower top. The piping 106, which has an end connected to the gas outlet of the heat exchanger 4, is connected to the gas inlet. Furthermore, a piping 107 is connected to the medium inlet, a piping 114 is connected to the medium outlet, and a piping 108 is connected to the gas outlet of the absorption tower 5.

The step (D) will specifically be described. The produced gas that has been subjected to the step (C), that is, the produced gas discharged from the heat exchanger 4 is fed to the absorption tower 5 through the piping 106, and in synchronization with this feeding, the absorption solvent is supplied to the absorption tower 5 through the piping 107. Thus, the absorption solvent is brought into countercurrent contact with the produced gas that has been subjected to the step (C), and so the other gases containing 1,3-butadiene in the produced gas, which has been subjected to the step (C), are selectively absorbed by the absorption solvent. As a result, the other gases containing 1,3-butadiene, and the molecular oxygen and the inert gases are roughly separated. While the absorption solvent, which has absorbed the other gases containing 1,3-butadiene, is discharged from the absorption tower 5 to the piping 114, the molecular oxygen and the inert gases, which have not been absorbed by the absorption solvent, are discharged from the absorption tower 5 to the piping 108.

The temperature inside the absorption tower 5 during operation is not particularly limited. In general, molecular oxygen and inert gases are hardly absorbed by the absorption solvent as the temperature inside the absorption tower 5 increases. On the other hand, the absorption efficiency of hydrocarbons such as butadiene (the other gases containing 1,3-butadiene) into the absorption solvent increases as the temperature inside the absorption tower 5 decreases. Thus, the temperature inside the absorption tower 5 may preferably be not lower than 0° C. and not higher than 60° C., more preferably not lower than 10° C. and not higher than 50° C., in consideration of the productivity of butadiene.

In addition, it is preferable that the pressure in the absorption tower 5 during operation (specifically, the pressure at the gas outlet of the absorption tower 5), that is, the pressure in the step (D) is equal to or less than the pressure in the step (C).

Specifically, the difference between the pressure in the step (D) and the pressure in the step (C), that is, a value obtained by subtracting the pressure in the step (D) from the pressure in the step (C) may preferably be 0 MPaG or more and not more than 0.05 MPaG, more preferably not less than 0.01 MPaG and not more than 0.04 MPaG.

When the pressure difference between the step (C) and the step (D) is controlled to fall within the aforementioned range, absorption of butadiene (the other gases containing 1,3-butadiene) into the absorption solvent in the absorption tower 5 can be promoted. As a result, the amount of the absorption solvent used can be reduced and energy consumption can be reduced.

Absorption Solvent:

Examples of the absorption solvent include those containing an organic solvent as a main component. As used herein, “containing an organic solvent as a main component” indicates that the content ratio of the organic solvent in the absorption solvent is not less than 50% by mass.

Examples of the organic solvent constituting the absorption solvent include an aromatic compound such as toluene, xylene and benzene, an amide compound such as dimethylformamide and N-methyl-2-pyrrolidone, a sulfur compound such as dimethyl sulfoxide and sulfolane, a nitrile compound such as acetonitrile and butyronitrile, and a ketone compound such as cyclohexanone and acetophenone.

The amount used (amount supplied) of the absorption solvent is not particularly limited, and may preferably be not less than 10 times by mass and not more than 100 times by mass, more preferably not less than 17 times by mass and not more than 35 times by mass, relative to the flow rate (mass flow rate) of the sum of butadiene, and 1-butene and 2-butene in the produced gas that has been subjected to the step (C).

When the amount used of the absorption solvent is controlled to fall within the aforementioned range, the absorption efficiency of the other gases containing 1,3-butadiene can improve.

On the other hand, when the amount used of the absorption solvent is excessively large, the energy consumption, which is used in purification for the absorption solvent to be circulated and used, tends to increase. In addition, when the amount used of the absorption solvent is excessively small, the absorption efficiency of the other gases containing 1,3-butadiene tends to decrease.

The temperature (temperature at the solvent inlet) of the absorption solvent may preferably be not lower than 0° C. and not higher than 60° C., more preferably not lower than 0° C. and not higher than 40° C.

When the temperature of the absorption solvent is controlled to fall within the aforementioned range, the absorption efficiency of the other gases containing 1,3-butadiene can further improve.

Circulation Step:

In the circulation step, the molecular oxygen and the inert gases obtained in the step (D) are fed as a reflux gas to the step (A). In this circulation step, the molecular oxygen and the inert gases from the step (D) are treated by a solvent-collecting tower 6 and a compressor 7.

This circulation step will specifically be described. The molecular oxygen and the inert gases from the step (D), that is, the molecular oxygen and the inert gases discharged from the absorption tower 5 are fed to the solvent-collecting tower 6 through the piping 108, subjected to a solvent removal treatment, and then fed to the compressor 7 through a piping 111. As necessary, a pressure adjustment treatment is performed. The molecular oxygen and the inert gases from the step (D) that have been subjected to the solvent removal treatment and the pressure adjustment treatment as described above are discharged from the compressor 7 to the piping 113 toward the reaction tower 1.

In the example of this drawing, while the molecular oxygen and the inert gases that have been discharged from the solvent-collecting tower 6 pass through the piping 111, a part of the molecular oxygen and the inert gases is discarded through a piping 112, which communicates with the piping 111. When the piping 112 for discarding a part of the molecular oxygen and the inert gases discharged from the solvent-collecting tower 6 is thus provided, the amount of the reflux gas to be supplied to the step (A) can be adjusted.

Solvent-Collecting Tower:

The solvent-collecting tower 6 is configured to wash the molecular oxygen and the inert gases from the step (D) with water or a solvent to perform the solvent removal treatment of the molecular oxygen and the inert gases. In the solvent-collecting tower 6, a gas inlet for introducing the molecular oxygen and the inert gases from the step (D) is provided at a central portion. A washing liquid inlet for introducing water or a solvent is provided at an upper portion of the solvent-collecting tower 6. The piping 108 having an end connected to the gas outlet of the absorption tower 5 is connected to the gas inlet, and a piping 109 is connected to the washing liquid inlet. In the solvent-collecting tower 6, a gas outlet for discharging the molecular oxygen and the inert gases washed with water or the solvent is provided at a tower top. In the solvent-collecting tower 6, a washing liquid outlet for discharging the water or solvent used in washing the molecular oxygen and the inert gases from the step (D) is provided at a tower bottom. The piping 111 is connected to the gas outlet of the solvent-collecting tower 6, and a piping 110 is connected to the washing liquid outlet of the solvent-collecting tower 6.

In this solvent-collecting tower 6, the absorption solvent contained in the molecular oxygen and the inert gases from the step (D) is removed, and the thus removed absorption solvent is discharged from the washing liquid outlet to the piping 110 together with the water or solvent used for washing, so as to be collected through this piping 110. Furthermore, the molecular oxygen and the inert gases from the step (D), which have been subjected to the solvent removal treatment, are discharged from the gas outlet of the solvent-collecting tower 6 to the piping 111.

In addition, the temperature inside the solvent-collecting tower 6 during operation is not particularly limited, and may preferably be not lower than 0° C. and not higher than 80° C., more preferably not lower than 10° C. and not higher than 60° C.

Compressor:

As the compressor 7, a compressor that is capable of increasing the pressure of the molecular oxygen and the inert gases from the solvent-collecting tower 6, as necessary, and adjusting the pressure to a pressure required in the step (A) is appropriately used.

In the example of this drawing, the compressor 7 has a gas inlet to which the piping 111, which has an end connected to the gas outlet of the solvent-collecting tower 6, is connected and a gas outlet to which the piping 113 is connected.

When the pressure in the step (D) is lower than the pressure in the step (A), pressurization by a pressure difference between the step (D) and the step (A) is performed by this compressor 7 according to the pressure difference.

When the pressurization is performed by this compressor 7, the pressure increase is usually small. Therefore, the electric energy consumption of the compressor is kept small.

In the molecular oxygen and the inert gases discharged from the compressor 7, that is, in the reflux gas, the concentration of molecular nitrogen may preferably be not less than 87% by volume and not more than 97% by volume, more preferably not less than 90% by volume and not more than 95% by volume. Furthermore, the concentration of the molecular oxygen may preferably be not less than 1% by volume and not more than 6% by volume, more preferably not less than 2% by volume or more and not more than 5% by volume.

Desolvation Step:

In the desolvation step, the absorption solvent, which is obtained in the step (D) and has absorbed the other gases containing 1,3-butadiene, is subjected to a treatment of separating a solvent. That is, when the absorption solvent is separated from the absorption solvent from the step (D), the other gases containing 1,3-butadiene, that is, a gas stream of the gas containing 1,3-butadiene is obtained. In the desolvation step, separation of the other gases containing 1,3-butadiene and the absorption solvent is performed by a desolvation tower 8 as illustrated in FIG. 1.

The desolvation step will specifically be described. The absorption solvent from the step (D), that is, the absorption solvent which has been discharged from the absorption tower 5 and has absorbed the other gases containing 1,3-butadiene is fed to the desolvation tower 8 through the piping 114 and is subjected to the treatment of separating a solvent. In the desolvation tower 8, the other gases containing 1,3-butadiene and the absorption solvent are separated by distillation.

Desolvation Tower:

The desolvation tower 8 is configured to perform a treatment of separating a solvent in which the absorption solvent from the step (D) is separated by distillation. A solvent inlet for introducing the absorption solvent from the step (D) is provided at a central portion of the desolvation tower 8. A gas outlet for discharging the gas containing 1,3-butadiene which has been separated from the absorption solvent from the step (D) is provided at a tower top of the desolvation tower 8. A solvent outlet for discharging the absorption solvent which has been separated from the absorption solvent from the step (D) is provided at a tower bottom of the desolvation tower 8. The piping 114, which has an end connected to the solvent outlet of the absorption tower 5, is connected to the solvent inlet of the desolvation tower 8, a piping 117 is connected to the gas outlet of the desolvation tower 8, and a piping 115 is connected to the solvent outlet.

In this desolvation tower 8, the absorption solvent, which has absorbed the gas, from the step (D) is separated into the gas containing 1,3-butadiene and the absorption solvent. Then, the gas containing 1,3-butadiene is discharged from the gas outlet to the piping 117, and the absorption solvent is discharged from the solvent outlet to the piping 115.

The pressure inside the desolvation tower 8 is not particularly limited and the pressure may preferably be not less than 0.03 MPaG and not more than 1.0 MPaG, more preferably not less than 0.2 MPaG and not more than 0.6 MPaG.

The temperature at the tower bottom of the desolvation tower 8 during operation may preferably be not lower than 80° C. and not higher than 190° C., more preferably not lower than 100° C. and not higher than 180° C.

According to such a method for producing 1,3-butadiene, the produced gas is washed in the step (B) before it is subjected to the step (C) of cooling the produced gas, to remove the solid component which would be deposited. Therefore, attachment of a large amount of the solid component, which is deposited by cooling in the step (C), to a solid packing, a tray, and the like in the quench tower 3 can be prevented or suppressed. Accordingly, the produced gas can be cooled stably for an extended period of time.

### EXAMPLES

Hereinafter, specific examples of the present invention will be described, but the present invention is not limited to these examples.

### Example 1

In accordance with the flow diagram of FIG. 1, 1,3-butadiene was continuously produced from a raw material gas containing 1-butene and 2-butene for 72 hours through the following steps (A), (C), and (D), desolvation step and circulation step.

As the raw material gas, a raw material gas containing 1-butene and 2-butene at a proportion of 2-butene of 87% by volume per 100% by volume of a sum of 1-butene and 2-butene was used.

Step (A):

To the reactor 1 (inner diameter: 21.2 mm, outer diameter: 25.4 mm) filled with a metal oxide catalyst so that the length of a catalyst layer was 1,500 mm, a mixed gas with a volume ratio (1-butene and 2-butene/O₂/N₂/H₂O) of 1/1.5/16.3/1.2 was supplied at a GHSV of 2,000 h⁻¹. The raw material gas and a molecular oxygen-containing gas were subjected to an oxidative dehydrogenation reaction under a condition of a reaction temperature of 320 to 350° C., to obtain a produced gas containing 1,3-butadiene. The pressure in this first step, that was, the pressure at the gas inlet of the reactor 1 was 0.1 MPaG. Herein, the GHSV based on actual volume of the mixed gas was 2,150 h⁻¹.

In this first step, as the metal oxide catalyst, a metal oxide catalyst in which an oxide represented by a composition formula: Mo₁₂Bi₅Fe_{0.5}Ni₂Co₃K_{0.1}Cs_{0.1}Sb_{0.2} was carried on spherical silica at a proportion of 20% of the whole catalyst volume was used.

As the mixed gas, the raw material gas and a reflux gas (molecular oxygen and inert gases) were mixed, and as necessary, air as the molecular oxygen-containing gas, molecular nitrogen as the inert gases, and water (water vapor) were further mixed, to adjust its composition.

Step (B):

The produced gas was washed under the following conditions using the washing unit illustrated in FIG. 2.

Mass ratio of washing liquid W to produced gas in washing unit 2: 1.1

pH of washing liquid W: 3 to 5

Step (C):

A quench tower having the following specification was used as the quench tower 3.

Total length of tower body 20: 0.4 cm

Inner diameter of tower body 20: 0.026 cm

Member for gas-liquid contact 37: random packing (raschig ring)

The number of random packings placed in member for gas-liquid contact 37: 270

The produced gas discharged from the reactor 1 was brought into countercurrent contact with a cooling medium in the quench tower 3 to be quenched, and cooled to 76° C. After that, the produced gas was cooled to 30° C. in the heat exchanger for cooling 4. The pressure in this step (C), that was, the pressure at the gas outlet of the quench tower 3 was 0.1 MPaG, and the pressure at the gas outlet of the heat exchanger for cooling 4 was also 0.1 MPaG.

Step (D):

The produced gas discharged from the heat exchanger 4 (hereinafter, also referred to as “cooled produced gas”) was supplied from the gas inlet, at the lower portion, of the absorption tower 5 (outer diameter: 152.4 mm, height: 7,800 mm, material: SUS304) inside of which a regular packing was disposed, and an absorption solvent containing toluene in an amount of not less than 95% by mass was supplied at 10° C. from the solvent inlet of the absorption tower 5 at the upper portion thereof. The amount of the absorption solvent supplied was 33 times by mass the flow rate (mass flow rate) of the sum of butadiene, and 1-butene and 2-butene in the cooled produced gas. The pressure in this step (D), that was, the pressure at the gas outlet of the absorption tower 5 was 0.1 MPaG.

Circulation Step:

The gas discharged from the absorption tower 5 was washed with water or a solvent in the solvent-collecting tower 6, to remove a small amount of absorption solvent contained in the gas. The gas in which the absorption solvent was thus removed was discharged from the solvent-collecting tower 6, a part of the gas was discarded, and most of the rest was fed to the compressor 7. In the compressor 7, the gas from the solvent-collecting tower 6 was pressurized by a pressure adjustment treatment. The absorption solvent was thus removed, and the pressurized gas was discharged from the compressor 7 and returned to the reactor 1.

Desolvation Step:

A liquid that had been discharged from the absorption tower 5 was supplied to the desolvation tower 8, and a gas that had been discharged from a body of the desolvation tower was cooled in a condenser, to obtain a gas containing 1,3-butadiene. A discharged liquid in which a part of a liquid that had been discharged from the body of the desolvation tower was heated by a reboiler, that was, an absorption solvent (hereinafter also referred to as “circulating absorption solvent”) was also obtained. Thus, the gas containing 1,3-butadiene and the circulating absorption solvent were separated in the desolvation tower 8 by distillation.

### Example 2

The same procedures as those of Example 1 were performed to continuously produce 1,3-budadiene from the raw material gas containing 1-butene and 2-butene for 72 hours except that in the step (B), the mass ratio of the washing liquid W to the produced gas in the washing unit 2 was changed to 8.9 and the pH of the washing liquid W was changed to fall within a range of 6 to 8.

### Comparative Example 1

The same procedures as those of Example 1 were performed to continuously produce 1,3-budadiene from the raw material gas containing 1-butene and 2-butene for 72 hours except that the step (B) was not performed.

In Examples 1 to 2 and Comparative Example 1, a lowermost member for gas-liquid contact 37 in the quench tower 3 was checked after completion of production of 1,3-butadiene. It was confirmed that a small amount of solid component was attached to the member in Example 1 whereas a large amount of solid component was attached to the member in Comparative Example 1.

The mass of the solid component deposited per 1 hour in each of the washing unit 2 and the quench tower 3 was measured, and the ratio of the mass of the solid component deposited in the washing unit 2 to the total mass of solid components deposited in the washing unit 2 and the quench tower 3 was determined as a removal rate of the solid component by the washing unit 2. As a result, the removal rate of the solid component in Example 1 was 59.4%, and the removal rate of the solid component in Example 2 was 72.9%.

From Examples 1 and 2, it is understood that attachment of a large amount of the solid component deposited by cooling to the members for gas-liquid contact 37 can be prevented or suppressed, and the produced gas can be cooled stably for an extended period of time.

### REFERENCE SIGNS LIST

1 reactor

2 washing unit

3 quench tower

4 heat exchanger

5 absorption tower

6 solvent-collecting tower

7 compressor

8 desolvation tower

20 chamber

20a scrubbing part

21 gas inlet

22 gas outlet

24 flow channel-forming member

25 vertical plate

26 first curved plate

27 second curved plate

28 auxiliary guide plate

29 washing-liquid outlet

30 tower body

31 peripheral wall portion

32 tower top

33 tower bottom

34 gas inlet

35 gas outlet

36 cooling-medium outlet

37 member for gas-liquid contact

38 mechanism for supplying cooling medium

39 spraying head

40 washing-liquid sprayer

100 to 117 piping

W washing liquid

1. A method for producing 1,3-butadiene, comprising:
(A) performing an oxidative dehydrogenation reaction between oxygen and a raw material gas comprising n-butene in the presence of a metal oxide catalyst, thereby obtaining a produced gas containing 1,3-butadiene;
(B) washing the produced gas obtained in (A);
(C) contacting the produced gas washed in (B) with a cooling medium to cool the produced gas; and
(D) separating the produced gas cooled in (C) into molecular oxygen and inert gases, and other gases containing 1,3-butadiene, by selective absorption into an absorption solvent, wherein
in (B), the washing of the produced gas includes blowing the produced gas onto a liquid surface of a washing liquid such that the produced gas contacts the liquid surface of the washing liquid.

2. The method for producing 1,3-butadiene according to claim 1, wherein (B) is performed in a washing unit, and a mass ratio of the washing liquid to the produced gas in the washing unit is 1.1 to 8.9.

3. The method for producing 1,3-butadiene according to claim 2, wherein the washing unit comprises at least one stage of a scrubbing part for washing the produced gas.

4. The method for producing 1,3-butadiene according to claim 1, wherein a pH of the washing liquid in (B) is within a range of 6 to 10.

5. The method for producing 1,3-butadiene according to claim 1, wherein the washing liquid after the washing of the produced gas in (B) is used as the cooling medium for cooling the produced gas in (C).

6. The method for producing 1,3-butadiene according to claim 1, wherein the washing liquid comprises water or alkaline water.

7. The method for producing 1,3-butadiene according to claim 1, wherein the washing liquid has a temperature of from 5° C. to 50° C.

8. The method for producing 1,3-butadiene according to claim 1, wherein the washing liquid has a temperature of from 5° C. to 30° C.

9. The method for producing 1,3-butadiene according to claim 1, wherein in (B), the washing of the produced gas includes blowing the produced gas onto the liquid surface of the washing liquid such that the liquid surface of the washing liquid is pushed down by pressure applied by the blowing of the produced gas.

10. The method for producing 1,3-butadiene according to claim 1, wherein in (B), the washing of the produced gas includes blowing the produced gas onto the liquid surface of the washing liquid such that the produced gas contacts the liquid surface of the washing liquid and that a by-product in the produced gas is deposited in the washing liquid as a solid component.

11. The method for producing 1,3-butadiene according to claim 3, wherein the scrubbing part comprises a flow channel for flowing the produced gas.

12. The method for producing 1,3-butadiene according to claim 1, wherein in (C), the produced gas is cooled to a temperature between 30° C. and 90° C. by the contacting of the produced gas with the cooling medium.

13. The method for producing 1,3-butadiene according to claim 1, wherein the absorption solvent comprises not less than 50% by mass of an organic solvent.

14. The method for producing 1,3-butadiene according to claim 1, wherein in (D), the other gases containing 1,3-butadiene comprises 1,3-butadiene, 1-butene, and 2-butene.

15. The method for producing 1,3-butadiene according to claim 1, wherein the absorption solvent is capable of absorbing the other gases containing 1,3-butadiene.

16. The method for producing 1,3-butadiene according to claim 1, wherein the metal oxide catalyst is a composite oxide catalyst comprising molybdenum and bismuth.

17. The method for producing 1,3-butadiene according to claim 1, wherein the an oxidative dehydrogenation reaction is performed at a pressure of 0.1 MPaG to 0.4 MPaG.

18. The method for producing 1,3-butadiene according to claim 1, wherein the an oxidative dehydrogenation reaction is performed at a temperature of 300° C. to 400° C.
